(19) United States
(12) Patent Application Publication
(10) Pub. No.: US 2006/0036204 A1
(43) Pub. Date: Feb. 16, 2006

(54) Ankle locking device

(76) Inventor: Ismael Armando Corrales, Lynchburg, VA (US)

Correspondence Address: NATIONAL IP RIGHTS CENTER, LLC;SCOTT J. FIELDS, ESQ., 550 TOWNSHIP LINE ROAD, SUITE 400, BLUE BELL, PA, 19422, US

(21) Appl. No.: 11/199,946

(22) Filed: Aug. 5, 2005

Related U.S. Application Data
(60) Provisional application No. 60/599,490, filed on Aug. 6, 2004.

(51) Int. Cl. A61F 5/00

(52) U.S. Cl. 602/27; 602/23; 128/882

(57) ABSTRACT

A device for improving kicking strength and accuracy by locking an athlete's ankle into proper kicking position comprising: a strap for attachment below the knee of a player; a tube affixed to the strap; an elastic cord inserted into a tube and affixed over the back cleats of the player; and a slidable locking connector for adjusting the tension of the cord.

### CLAIM OF PRIORITY

This application claims benefit to provisional application 60/599,490, filed on Aug. 6, 2004, which is incorporated by reference herein in its entirety.

### FIELD OF THE INVENTION

The present invention is directed to a device and system which assists in locking an athlete's ankles, which can increase strength and accuracy when kicking a ball.

### BACKGROUND OF THE INVENTION

In sports such as soccer, it is very important for the athlete to lock his or her ankle when kicking a ball. When the ankle is locked, the hardest part of the foot is exposed, thus maximizing kicking power and accuracy. There have been a number of patents directed to foot support systems. U.S. Pat. No. 6,695,797 B2 discloses a foot support system that includes a strap that wraps around the leg, a strap that wraps around the foot, and an elastic member extending between the two straps.

U.S. Pat. No. 5,291,904, shows a dorsiflexion assisting device having a strap that wraps around the calf of the wearer, another strap that wraps around the ankle region of the wearer, an elastic connecting the calf strap and the ankle strap, and a cord attached to the ankle strap and the outer bottom of the shoe.

U.S. patent application U.S. 20050043121 A1 describes a training apparatus for athletes that includes a belt or strap that fits around the user's waist, an ankle strap, and a strap extending between the belt and the ankle strap. A portion of the strap extending between the belt and the ankle strap may be elastic.

It is an object of the present to provide a device for locking the athlete's ankle to increase kicking strength and accuracy.

It is a further object of the invention to provide a device for improving ligament, tendon, calf and shin muscle strength.

These and other objects of the present invention have been described and illustrated.

### SUMMARY OF THE INVENTION

A first embodiment of the invention comprises a device for improving kicking strength and accuracy by locking the athlete's ankle into proper position comprising: a strap for attachment below the knee of a player; a tube affixed to the strap; an elastic cord inserted into a tube and affixed over the back cleats of the player; and a slidable locking connector for adjusting the tension of the loop.

Another embodiment of the invention comprises an ankle, ligament, tendon, calf and shin muscle strengthening device comprising: a strap for attachment below the knee of a player; a tube affixed to the strap; an elastic band inserted into a tube and affixed over the back or front part of the shoe; and a slidable locking connector.

Yet another embodiment of the invention comprises an ankle strengthening device comprising: a strap for attachment below the knee of a player; a tube affixed to the strap; an elastic loop inserted into a tube and affixed over the back cleats of the player; and a slidable locking connector for adjusting the tension of the loop.

### BRIEF DESCRIPTION OF THE DRAWINGS

FIG. 1 is an elevational view of the present invention.

FIG. 2 is an elevational view of the present invention attached to the wearer.

FIG. 3 is a view of the present invention attached to the heal of a player.

FIG. 4 illustrates the device being adjusted.

FIGS. 5 to 7 illustrate an alternative embodiment of the present invention.

### DESCRIPTION OF THE PREFERRED EMBODIMENT

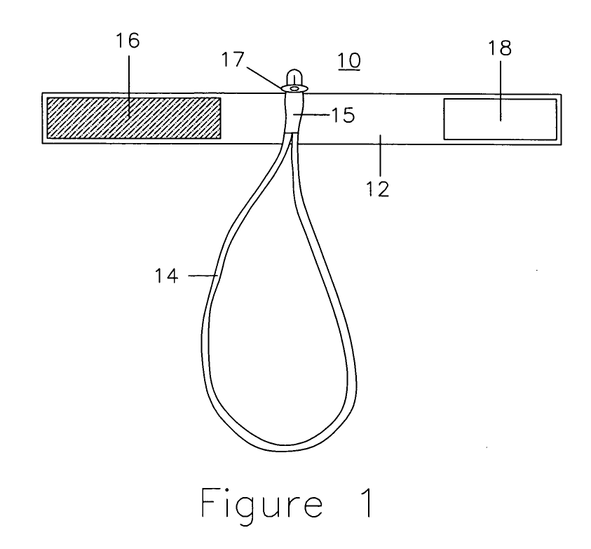

The present invention is directed to a device and apparatus for improving kicking strength and accuracy by locking the ankle of the athlete. The invention is now described. Referring to FIG. 1, the invention 10 comprises a neoprene strap 12 which has a Velcro attachment 16, 18 to secure to the player just below the knee. A loop 10 of an elastic material such as bungee cord is inserted into a fabric tube 15. The top of the cord is affixed to a slidable locking connector 17.

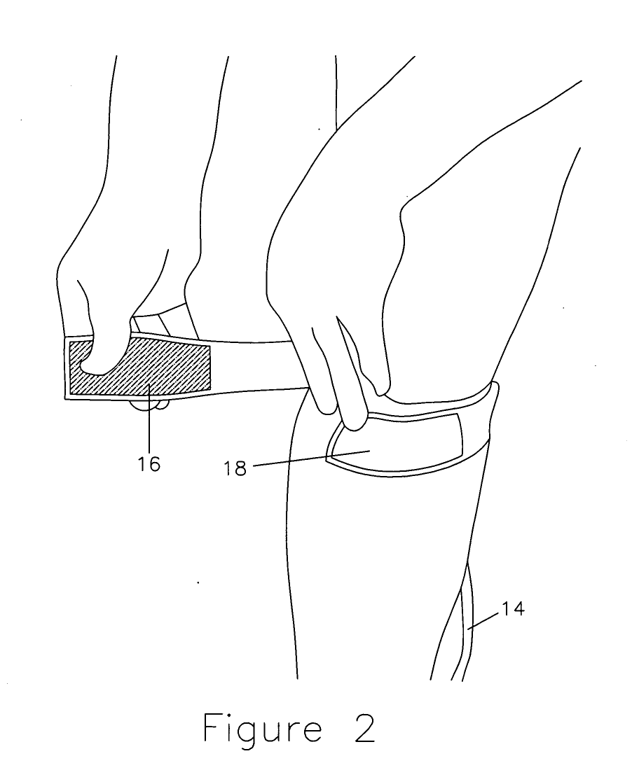

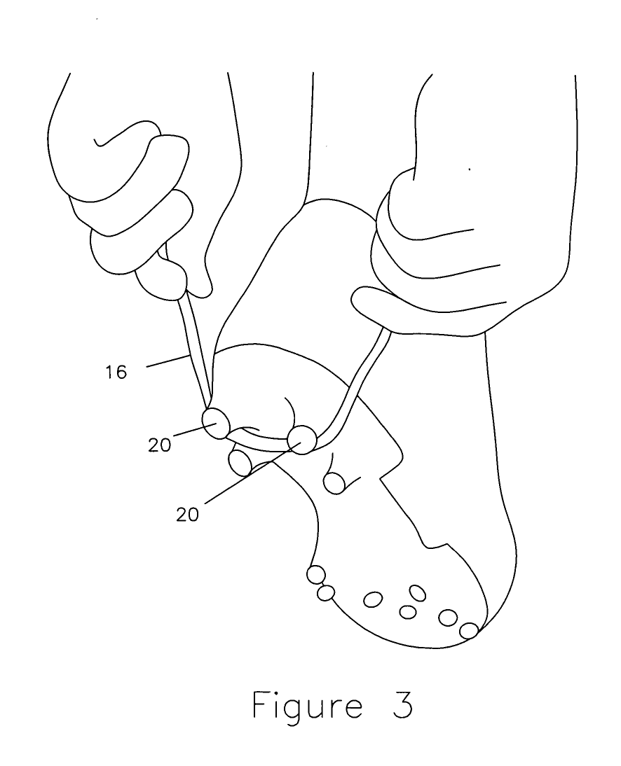

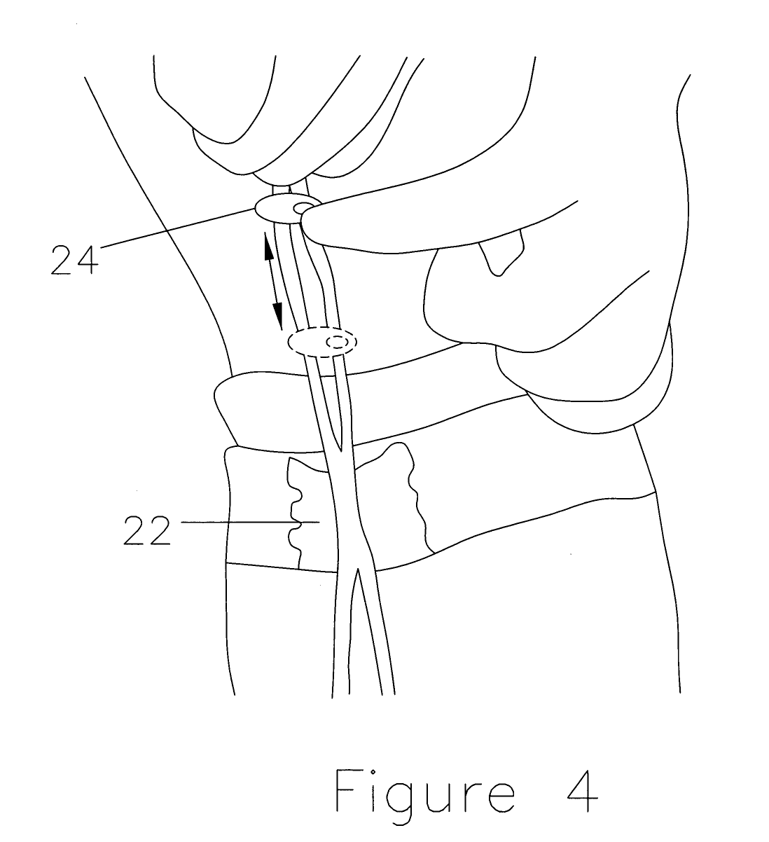

Referring to FIGS. 2-4, the operation of the invention is now described. First, the strap 12 is affixed snugly below the user's knee. The loop end 16 is affixed over the back cleats 20 of a pair of cleats. The locking connector 17 is then adjusted to increase or decrease tension on the wearer.

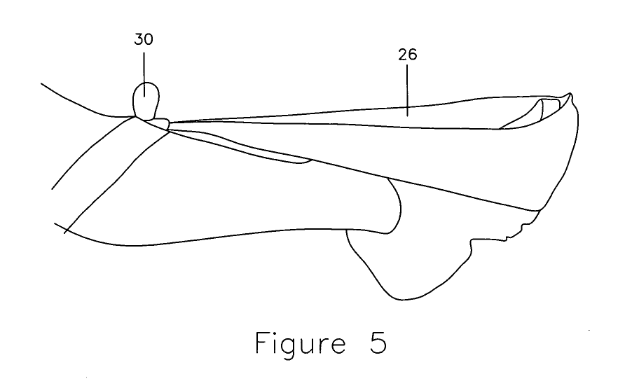

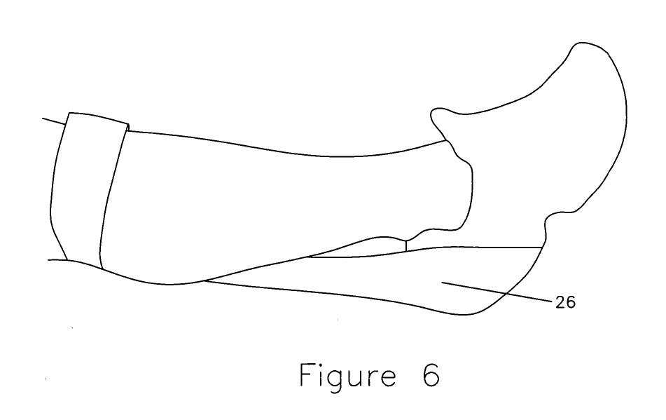

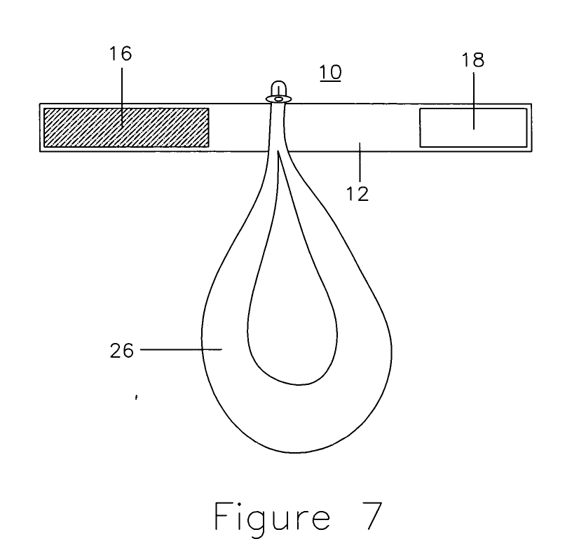

Referring to FIGS. 5-7, an alternative embodiment is shown. As shown in FIG. 7, the alternative embodiment of the invention comprises a thicker band. This band is constructed from a material such as TheraBand, manufactured by the Hygienic Corporation. The band can be used for both the forward and rear of the foot as shown in FIGS. 5 and 6. Both embodiments can be utilized in physical rehabilitation and sports medicine. The present invention has been described with reference to the above discussed preferred embodiment. It is to be appreciated that the true nature and scope of the invention is to be determined with reference to the claims appended hereto.

1. A device for improving kicking strength and accuracy by locking an athlete's ankle into proper position comprising:
a strap for attachment below the knee of a player; a tube affixed to the strap;
an elastic cord inserted into a tube and affixed over the back cleats of the player; and a slidable locking connector for adjusting the tension of the cord.

2. The ankle locking device of claim 1 wherein the strap is constructed from neoprene.

3. The ankle locking device of claim 1 wherein the strap is attached by a Velcro connector.

4. An ankle locking device comprising:
a strap for attachment below the knee of a player;
a tube affixed to the strap;
an elastic cord inserted into a tube and affixed over the back or front cleats of the player; and
a slidable locking connector for adjusting the tension of the cord.

5. An ankle locking device comprising:
a strap for attachment below the knee of a player;
a tube affixed to the strap;
a theraband cord inserted into a tube and affixed over the back or front cleats of the player; and
a slidable locking connector for adjusting the tension of the cord.
